(19) United States
(12) Patent Application Publication
(10) Pub. No.: US 2026/0103740 A1
(43) Pub. Date: Apr. 16, 2026

(54) METHOD AND APPARATUS FOR USING LYOPHILIZED REAGENT BEADS FOR ANTIMICROBIAL SUSCEPTIBILITY TESTING

(71) Applicants: General Electric Company, Schenectady, NY (US); The Johns Hopkins University, Baltimore, MD (US)

(72) Inventors: Christopher Michael Puleo, Niskayuna, NY (US); Ralf Lenigk, Schnectady, NY (US); Tejas Suresh Khire, Niskayuna, NY (US); Brian Christopher Bales, Niskayuna, NY (US); Gregory Andrew Grossmann, Halfmoon, NY (US); Sara Kelly Peterson, Schenectady, NY (US); Tza-Huei Wang, Germantown, MD (US); Alexander Trick, Baltimore, MD (US); Fan-En Chen, Baltimore, MD (US)

(21) Appl. No.: 19/112,160

(22) PCT Filed: Sep. 15, 2023
(86) PCT No.: PCT/US2023/032935

§ 371 Date: Mar. 14, 2025

Related U.S. Application Data
(60) Provisional application No. 63/375,810, filed on Sep. 15, 2022.

(51) Int. Cl. C12Q 1/18; C12M 1/00; C12M 1/34; C12M 3/06; C12Q 1/686; C12Q 1/689

(52) U.S. Cl. CPC C12Q 1/18; C12M 23/16; C12M 23/34; C12M 23/40; C12M 29/20; C12M 41/12; C12Q 1/686; C12Q 1/689

(57) ABSTRACT

The present disclosure is directed towards using lyophilized reagent beads for a fully closed multi-step reaction to complete antibiotic susceptibility tests and amplification assays. The lyophilized reagent beads include a lyophilized amplification reagent bead and an lyophilized antibiotic bead. The lyophilized reagent bead may be embedded in solid wax layer inside the test tube to enable cell growth in antibiotic prior to release of amplification reagents. A microfluidic manifold device may be used to insert the sample into the amplification tube and enable a fully closed system.

### CROSS REFERENCE TO RELATED APPLICATIONS

The present application claims the benefit of priority to U.S. Provisional Application No. 63/375,810, filed on Sep. 15, 2022, and also claims priority to International Application No. PCT/US2023/032935, filed on Sep. 15, 2023. Both applications are incorporated herein by reference in their entirety.

### STATEMENT REGARDING FEDERALLY SPONSORED RESEARCH & DEVELOPMENT

This invention was made with government support under grant AI138978 awarded by the National Institutes of Health and MCDC Base Agreement 2018-342 and the MCDC Project Agreement MCDC-18-01-01-012. The government has certain rights in the invention.

### BACKGROUND

The subject matter disclosed herein relates to methods and apparatuses for using lyophilized reagent beads to test susceptibility of microbes to the effect of antibiotics through closed-system antimicrobial susceptibility testing (AST) and amplification-based assays.

Antimicrobial resistance (AMR) is an increasing issue as a result of over-use and incorrect use of antibiotic drugs. This has resulted in evolution of drug resistant bacteria strains over time. Current bacterial culture methods that include longer time to antibiotic administration have contributed to the rise in AMR. While the empirical use of antibiotics is inevitable in emergency settings, the existing delay between blood culturing and the knowledge of in vitro susceptibility data reduces the overall efficacy of this empirical treatment. As a result of this, methods that amplify bacterial genetic material are replacing traditional culture methods, but these nucleic acid amplification methods may provide little to no information in regards to phenotypic susceptibility to antibiotics.

### BRIEF DESCRIPTION

In one embodiment, a method of antibiotic susceptibility testing includes providing an amplification lyophilized reagent bead comprising amplification reagents, wherein the amplification lyophilized reagent bead is embedded within a first layer of a reaction vessel, and wherein the first layer comprises a phase change material having a melting point temperature. The method further includes providing an antibiotic bead comprising one or more antibiotics to the reaction vessel while the first layer is solid, and adding a bacterial sample to the reaction vessel to contact the antibiotic bead in a second layer. Additionally the method includes incubating contents of the reaction vessel at a temperature below the melting point temperature and to release the one or more antibiotics of the antibiotic bead in the second layer, increasing a temperature of the contents of the reaction vessel to be above the melting point temperature to melt the phase change material to permit the bacterial sample to contact the amplification lyophilized reagent bead, and conducting amplification analysis of the bacterial sample using amplification reagents of the amplification lyophilized reagent bead.

In an additional embodiment, a method of antibiotic susceptibility testing includes providing a reaction vessel including an amplification bead comprising lyophilized amplification reagents; a solid layer of a phase change material; and an antibiotic bead comprising one or more lyophilized antibiotics. Further, the method includes adding a bacterial sample to the reaction vessel to contact the antibiotic bead in a second layer to at least partially dissolve the antibiotic bead and form a liquid layer comprising the bacterial sample and the at least partially dissolved antibiotic bead separate from the solid layer, melting the solid layer to cause displacement of the liquid layer and such that the amplification bead dissolves in the liquid layer; and conducting an amplification assay of the bacterial sample using the amplification reagents of the amplification bead. The amplification bead may also contain such dyes (i.e. DNA intercalation dyes or labels) or detectable moieties that interact with the amplified material such that the increase in amplified matter is detectable.

In an additional embodiment, an antibiotic susceptibility testing reaction vessel is provided that includes an amplification bead comprising lyophilized amplification reagents embedded within a solid layer; and an antibiotic bead comprising one or more lyophilized antibiotics. The solid layer is solid below the incubation temperature of microbes and antibiotics bead in a range of 20 to 50° C. (e.g., 37° C.) and has a melting point below the range of the typical PCR annealing temperature, typically below 75° C. (or below the temperature for the isothermal amplification) such that the detection of amplified genetic material is not negatively affected by a solidified (opaque) layer. In an embodiment, the melting point is between 42° C. and 90° C.

### BRIEF DESCRIPTION OF THE DRAWINGS

These and other features, aspects, and advantages of the present disclosure will become better understood when the following detailed description is read with reference to the accompanying drawings in which like characters represent like parts throughout the drawings, wherein:

FIG. 1 is a flow diagram of a method of using lyophilized reagent beads to perform antimicrobial susceptibility testing (AST) testing, in accordance with embodiments of the present disclosure;

FIG. 2 is a schematic diagram of steps of performing AST testing and PCR assays using the lyophilized reagent beads, in accordance with embodiments of the present disclosure;

FIG. 3 is a graphical representation of lyophilized drug reagent bead testing during PCR assays, in accordance with embodiments of the present disclosure;

FIG. 4 is a graphical representation of bacteria growth using the lyophilized reagent drug bead, in accordance with embodiments of the present disclosure;

FIG. 5 is an image of a microfluidic manifold that includes one or more samples with the lyophilized reagent drug bead, in accordance with embodiments of the present disclosure;

FIG. 6 is a schematic diagram of the microfluidic manifold of FIG. 5, in accordance with embodiments of the present disclosure; and

FIG. 7 is a graphical representation of microfluidic manifold sample retention, in accordance with embodiments of the present disclosure.

### DETAILED DESCRIPTION

Antimicrobial resistance (AMR) is a growing global problem that results in evolution of multi-drug resistant and/or extreme-drug resistant bacterial strains. Current bacterial culture methods may result in longer time to antibiotic administration and contribute to the rise of AMR. Polymerase chain reaction (PCR) and other nucleic acid amplification methods are replacing the traditional culture methods to combat this issue. However, PCR and other nucleic amplification methods are mainly used to detect genotypic determinants of resistance and provide very little information on desired phenotypic susceptibility to antibiotics. This often makes nucleic acid amplification methods difficult to implement when determining phenotypic susceptibility to antibiotics.

In particular, nucleic acid amplification tests such as PCR have been replacing cell-culture methods to diagnose the presence of microbial infections in complex biological matrices. Amplification may provide some advantages over current cell-culture methods including providing less invasive sample collection methods, improved sample storage and transport conditions, the ability to process non-viable cells to increase test assay robustness, and increased speed of sample processing (e.g., a few hours) relative to traditional cell-culture methods. Traditionally, nucleic acid amplification has been implemented to detect genotypic AMR by determining genetic mutations associated with AMR. However, genotypic information is unable to be used to predict phenotypic resistance and is often susceptible to false positives due to detecting commensal pathogens over their infectious counterparts. Additionally, due to the reliance of clinical practitioners on nucleic acid amplification compared to culture-based AST testing, susceptibility data is not often available to physicians. Unfortunately, antimicrobial dosages below the minimum inhibitory concentration can increase rates of treatment failures and lead to resistant bacteria.

Provided herein are techniques for simultaneous implementation of both AST and amplification assays such as PCR or isothermal amplification for generating targeted antimicrobial administration or treatment recommendations in a reduced time period compared to traditional cell-culture methods. The two reaction reagents are spatially separated into different layers and can be temporally controlled, thus eliminating the need to transfer reagents across different consumables and minimizing user interactions.

To accomplish performance of bacteria cell growth and amplification analysis in the same reaction vessel, a lyophilized reagent mixture for AST and amplification is implemented. The lyophilized reagent mixture is prepared in a dry bead format and loaded into an amplification vial. The dry amplification bead is separated from the other contents of the amplification vial by a thermally activated barrier, e.g., a paraffin wax seal. Each reaction vessel may include a first bead containing an antibiotic drug for AST assay and a second bead containing the amplification reagents. The amplification bead is embedded in the solid wax that remains solid during a cell culture phase of the AST assay, but melts and releases nucleic acid amplification test reagents during the amplification phase. The wax helps shield the amplification bead from dissolution during the sample incubation step (bacteria growth phase) of the AST assay within the amplification tube. In this way, AST and amplification processes may be implemented within a single reaction vessel. The lyophilized reagent beads enable long-term storage and stabilization of sensitive biological reagents (e.g., antibiotic drugs) and further increases room temperature stability, reducing the possible occurrence of hydrolytic degradation of DNA primers and amplification enzymes during storage. The lyophilized antibiotic drugs in bead form also enable immediate dissolution upon sample addition for rapid activity of the antibiotic drug of interest. The multi-phase workflow using the amplification bead and the antibiotic bead enables phenotypic antimicrobial susceptibility testing and amplification assays to be completed in a closed system single-vessel format.

The disclosed techniques can be used in conjunction with a microfluidic manifold that enables equal volumetric sample distribution from a common input into one or more PCR or other amplification reaction vials. As discussed above, each of the amplification vials may be preloaded with different antibiotic and/or reagent beads enabling the multiplexed minimum inhibitory concentration analysis to be carried out using the microfluidic manifold. The microfluidic manifold may be designed to yield an output at each of its ends, uniformly distributing a bacterial load within the sample amongst the one or more test amplification vials. The microfluidic manifold increases throughput for screening antibiotics and minimum inhibitory concentration predictions based on the even sample distribution.

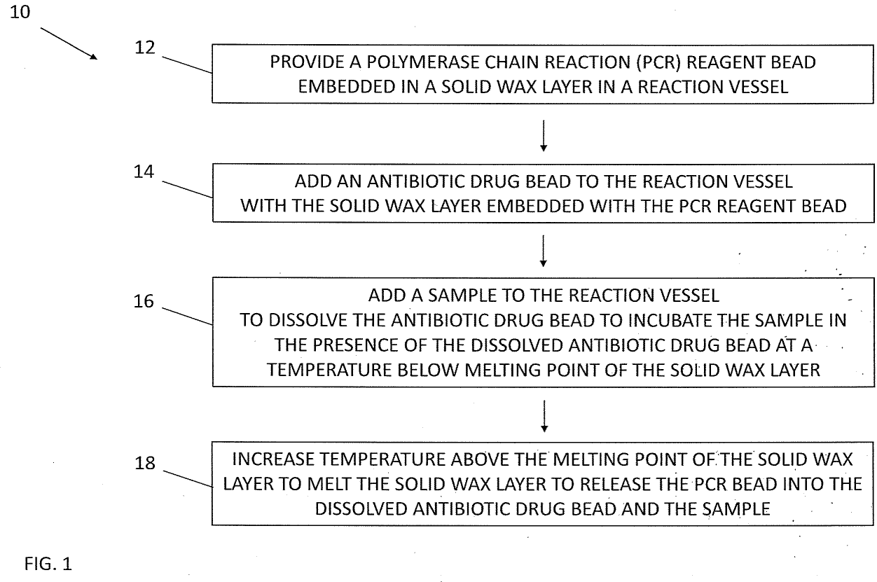

With the foregoing in mind, FIG. 1 is a flow diagram of a method 10 of using lyophilized reagent beads to perform AST testing and amplification analysis for multiplexed minimum inhibitory concentration analysis, in accordance with embodiments of the present disclosure. As discussed above, lyophilized beads may be pre-loaded into amplification vials to enable performing both bacterial cell growth and amplification analysis in the same reaction vessel. The amplification vials may include two lyophilized reagent beads, a first bead containing an antibiotic drug at a specified concentration and a second bead including an amplification reaction mixture. It should be understood, that while the term lyophilized reagent bead is used, the lyophilized reagent may be formed into any other suitable shape.

The method 10 provides, at block 12, an amplification bead (e.g., a PCR lyophilized reagent bead) embedded in a thermal barrier material such as a solid wax layer in a reaction vessel (e.g., amplification vial). The amplification bead may be embedded in a solid wax layer that includes a melting point of around 65° Celsius (C). The amplification reagents may include primers, enzymes, deoxyribonucleotide triphosphates (dNTPs), buffer salts, or any other suitable amplification reagent to be used for amplification analysis. The wax may act as a protectant for the amplification bead during an incubation and/or cell-culture phase of the test tube during AST. In an embodiment, the amplification bead may be embedded in wax or inert, thermally-activated phase change materials (e.g., polymers) that are solids at room temperature or solids at bacterial incubation temperatures (e.g., solid at 35° C.-40° C.) with a melting point below the primer annealing temperature of the amplification reaction, which may be above 65° C. or above 37-40° C. for certain isothermal amplification techniques. In an embodiment, the phase change material may include hydrated salts, paraffin waxes, fatty acids, or the eutectics of organic and non-organic compounds and polymers. In an embodiment, the amplification bead is embedded or provided in a material or wax having a melting point between 40° C. and 90° C. The solid wax or other layer acts to protect or segregate the amplification reagents of the amplification bead during bacterial incubation.

The method 10, at block 14, includes adding an antibiotic lyophilized reagent bead to the reaction vessel with the solid wax layer embedded with the amplification bead. The antibiotic bead (e.g., the antibiotic lyophilized reagent bead) may be stored on top of the wax layer. The antibiotic bead may include an antibiotic drug that is a drug of interest at a specified concentration for the multiplexed minimum inhibitory concentration analysis. The method 10, at block 16, includes adding a liquid-containing sample (e.g. bacterial sample) to the reaction vessel that dissolves the antibiotic bead to incubate the sample in the presence of the dissolved antibiotic drug at a temperature below the melting point of the solid wax layer. It should be understood that a bacteria sample may be a biological or environmental sample of interest that has an unknown composition that may include bacteria or other pathogens of interest. The melting point of the solid wax layer may be around 65° C. as discussed above, and the incubation may take place at about 37° C. or any other suitable range corresponding to incubation of the bacteria of interest being analyzed. The addition of the sample (e.g., bacteria) dissolves the antibiotic drug of the antibiotic bead immediately upon addition, and releases the antibiotic drug in the growth medium that may be added with the sample. In another embodiment, the growth medium can be provided in dried form together with the antibiotic bead. It should be understood that the incubation period may be any suitable period of time (e.g., 3-4 hours) for incubation of the bacterial cells in the presence of the dissolved antibiotic. In an embodiment, the incubation time may be less than conventional incubation times of 20-24 hours. However, the incubation time may vary depending on the microbe of interest.

The method 10, at block 18, includes increasing the temperature of the amplification reaction vessel above the melting point of the solid wax layer to melt the solid wax layer and release the amplification bead into the dissolved antibiotic drug and the sample after completion of the incubation period. The temperature increase may correspond to above 90° C., between 92° C.-98° C., or around 98° C., or any other suitable temperature that corresponds to initiation of the amplification reaction. The transition from around 37° C. to around 98° C. (e.g., denaturation temperature for amplification) melts the wax layer. The phase inversion of the cell sample to the bottom of the tube upon the melting of the wax, facilitates rapid dissolution of the amplification bead in the solution. The temperature of the amplification denaturation enables bacterial cell lysis and subsequent DNA release into the solution. It should be understood, that this method may be applied using any suitable temperature for bacterial incubation and/or amplification reaction. Further, while the disclosed embodiments are discussed in the context of amplification, additional or other amplification reactions are also contemplated, including ligation-mediated amplification, ligase chain reaction, or transcription-based amplification.

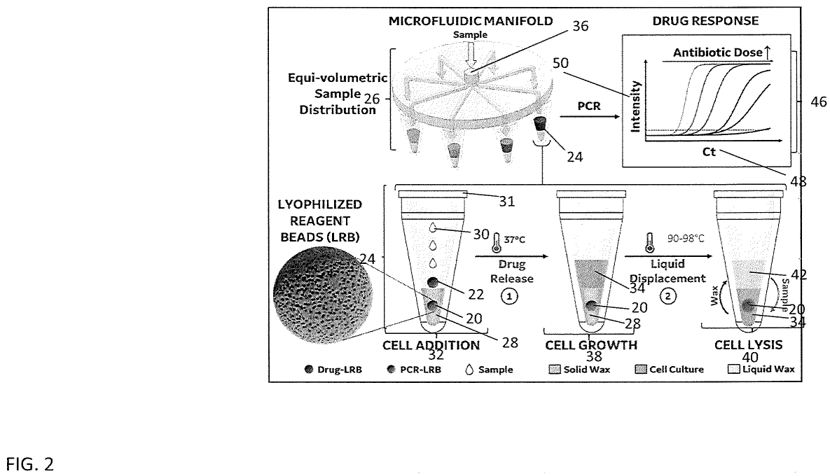

Keeping the foregoing in mind, FIG. 2 is a schematic diagram of steps of performing phenotypic antimicrobial susceptibility testing (AST) and amplification using the lyophilized reagent beads, in accordance with embodiments of the present disclosure. As discussed above the amplification beads 20 are lyophilized reagent beads that may be used with the antibiotic bead 22 to enable AST and amplification assays to be carried out in an amplification reaction vessel 24. The reaction vessel 24 may be a test tube, chamber, well, or other container. In an embodiment, the reaction vessel 24 may be configured to fluidically couple to a reaction system. For example, a microfluidic manifold 26 may be used for sample 30 distribution to enable equal distribution of samples 30 to each of the single amplification reaction vessels 24 used for AST and amplification testing and multiplexed MIC analysis. Thus, each reaction vessel 24 may include an end 31 that snaps into or fluidically couples (e.g., a one-way coupling) to an individual receiving end of the manifold 26 through which the sample is distributed.

For example, multiple amplification reaction vessels 24 may be attached to the microfluidic manifold 26 that may enable even sample 30 distribution from the microfluidic manifold sample inlet 36 to each of the amplification reaction vessels 24. Each of the amplification reaction vessels 24 may include different and/or similar antibiotics at varying concentrations for AST and amplification testing and analysis. Each amplification vial may include two lyophilized reagent beads. The first lyophilized regent bead may be an antibiotic bead 22 and the second lyophilized regent bead may be the amplification bead 20. The amplification bead 20 may include an amplification reaction mixture including a primer that may be used during amplification to enable DNA amplification for amplification assays. The amplification bead 20 may be formed by stabilizing amplification enzymes in a dried state at ambient temperatures and freeze-drying all the amplification components (e.g., buffer, required enzyme co-factors, dNTPs, primers and polymerase enzyme). The amplification bead 20 and the antibiotic bead 22 are both in a dried state. The stabilization of the lyophilized regent bead in the dried state eliminates precise liquid metering and distribution of reaction components during use of the lyophilized regent bead. Additionally, having all the reagents (e.g., amplification and antimicrobial drug) in the lyophilized state increases room temperature stability relative to liquid reagents, reducing the likelihood of hydrolytic degradation of DNA primers and amplification enzyme during storage of the lyophilized regent beads. In the dried state, the lyophilized reagent beads are highly porous and enable rapid dissolution in aqueous solutions. The lyophilized reagent beads include lyophilization-stabilizing components, which remain compatible with both the growth step of bacterial cells and the application step of bacterial DNA. For example, the amplification bead 20 solution may include Tricene, ammonium sulfate, magnesium chloride, dNTPs, forward and reverse primers, glycerol free solution, Tris-HCl, ethylenediamine tetraacetic acid (EDTA), and any other suitable amplification components. The lyophilized reagent bead format for stabilizing the amplification enzymes at ambient temperatures (e.g., room temperatures) in the amplification bead 20 may include freeze drying all amplification-components (e.g., buffer, enzyme co-factors, dNTPs, primers and polymerase, enzyme). The beads may be spherical or generally elliptical in form after lyophilization, but may also take the form of any suitable shape and/or particle configuration. The amplification bead 20 and/or an antibiotic bead 22 as provided herein may a regular or irregular shape, and different beads 20, 22 may have a generally same shape or size or different shapes and sizes. Further, while the disclosed embodiments are illustrated in the context of a single bead 20 and a single bead 22 per reaction vessel 24, it should be understood that, in an embodiment, multiple beads may be used. The antibiotic bead may have a lyophilized shell that is room temperature stable but that dissolves upon contact with the liquid in a bacterial sample.

The amplification bead 20 may be embedded within a solid wax 28 or other material as provided herein with a melting point above 40° C., above 65° C., or any other suitable melting point above the incubation temperature (e.g., 37° C. or 35° C. for certain organisms) for cell growth. The amplification bead 20 embedded within the solid wax 28 may be added to the amplification reaction vessel 24 during a cell addition phase 32, and may be located at the end of the amplification reaction vessel 24 distal to the opening of the amplification reaction vessel 24. The antibiotic bead 22 may be stored within the amplification reaction vessels 24 on top of the solid wax 28 layer as part of a device or added or provided into the vessel 24 together with the sample 30. One or more samples 30 may be added to each of the amplification reaction vessels 24 via the microfluidic manifold 26 as discusses above.

The sample 30 may include bacteria that may dissolve the antibiotic bead 22 upon insertion of the sample 30 into the amplification reaction vessels 24, and release the antibiotic drug into the growth medium added with the sample 30 via the microfluidic manifold 26. The amplification reaction vessels 24 with the released antibiotic drug in the growth medium are then incubated at 37° C. for an appropriate period of time according to the AST testing growth temperatures during a cell growth phase 38, to promote cell growth and enable cell culture 34 to be produced by the mixture of the growth medium, antibiotic bead 22, and the sample 30.

Following the incubation of the mixture, shown at 37° C. but other temperatures are also contemplated, the temperature of the amplification reaction vessels 24 is increased to above the solid wax 28 melting point (e.g., above 95° C. in an embodiment) during a cell lysis phase 40, to initiate the amplification reaction of the cell culture 34 with the amplification reagents in the amplification bead 20. The increased temperature corresponds to the solid wax 28 melting point, and results in melting of the solid wax 28. The phase change of the solid wax 28 from solid to liquid wax 42 reduces the wax density, and enables the liquid wax 42 to rise above the cell culture 34, and releases the amplification bead 20 into the cell culture 34. This release of amplification bead 20 into the cell culture 34 facilitates the dissolution of the amplification reaction mixtures with the cell culture 34, and promotes initiation of the amplification of the DNA released from the bacteria cells during autolysis as a result of the temperature increase (e.g., the bacteria can undergo autolysis at ˜95° C. or at temperatures used to initiate amplification).

The amplification reaction vessels 24 may then undergo amplification testing to determine bacteria growth as a result of bacteria within each amplification vial based on the varying concentrations of the antibiotic. Inhibition of growth and bacteria doubling as a result of the different concentrations of antibiotics, results in different concentrations of bacteria after the cell culture period, and prior to the wax melting and initiation of amplification caused by the temperature increase. The growth inhibition based on antibiotic concentration may be graphed as a drug response graph 46 that includes cycle threshold (Ct) 48 values on the x-axis, and antibiotic intensity 50 on the y-axis. The amplification reaction vessels 24 with less bacteria resulting from growth inhibition result in higher cycle threshold (Ct) values 48 after amplification testing and analysis. From this it may be determined the effects of certain antibiotics and concentration of antibiotics on bacteria growth.

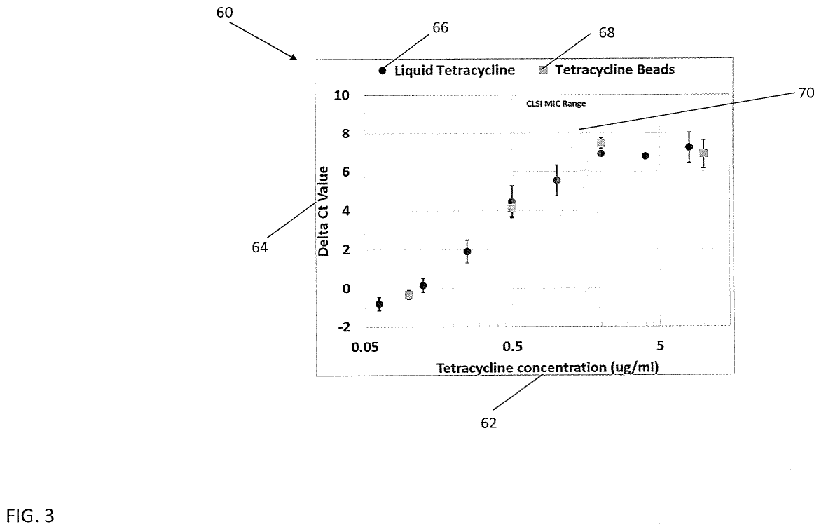

With the foregoing in mind, FIG. 3 is a graph of results of lyophilized drug reagent bead testing 60 during amplification assays, in accordance with embodiments of the present disclosure. amplification assays after growing bacteria (e.g., E. coli) in the presence of different concentrations of antibiotics (e.g., tetracycline hydrochloride) within the amplification reaction vessels 24 that include the antibiotic bead 22 were conducted, and the resulting Ct results were collected. The tetracycline-lyophilized reagent bead 68 was prepared such that each tetracycline-lyophilized reagent bead 68 when dissolved in 10 microliters of water yields a solution containing 0.1, 0.5, 2, and 10, micrograms of tetracycline/mL of water. To complete the trials, the embedded amplification bead 20 in the solid wax 28 was placed at the bottom of each of the amplification reaction vessels 24, and the antibiotic bead 22 was placed on top of the solid wax 28. An aqueous bacterial sample (e.g., e. coli in CAMH) was added to each of the amplification reaction vessels 24, and resulted in the antibiotic bead dissolving and releasing the tetracycline antibiotic in the solution to the bacterial cells. Bacterial growth in the presence of the varying tetracycline concentrations mentioned above was conducted for a period of about three hours at an incubation temperature of 37° C. The temperature was then raised to above the melting point of the solid wax 28, and amplification was performed to assess the inhibitory effects of tetracycline on bacterial growth. The threshold value (Ct) of the amplification reactions were measured. The higher Ct values imply lower DNA concentrations, and therefore drug-inhibited growth. The data was normalized by subtracting the mean Ct value for no-drug positive control from individual Ct values across the drug-containing incubations. The efficacy of the antibiotic bead 22 was verified based on running the same amplification assay in parallel with liquid tetracycline 66 at equivalent concentrations.

As shown in the graphical representation of lyophilized drug reagent bead testing 60, the tetracycline concentration 62 for both liquid tetracycline 66, the control group reagent, and tetracycline-lyophilized reagent beads 68 were measured in micrograms per milliliter (μg/mL), relative to the delta Ct values 64 produced by the concentration. The shaded region of the graph indicates the mean inhibitory concentration (MIC) values 70 as established by CLSI guidelines. The error bars are included to indicate standard deviation in the delta Ct values. For several antibiotic drug concentrations, the amplification reactions were conducted using both the liquid drug reagents 66 (e.g., tetracycline) and the lyophilized bead reagents 68 (e.g., tetracycline beads) during the amplification incubation discussed above for the amplification reaction vessels 24. It should be understood that the lyophilized bead reagents 68 yielded approximately the same delta Ct 64 values as the liquid drug reagents 66.

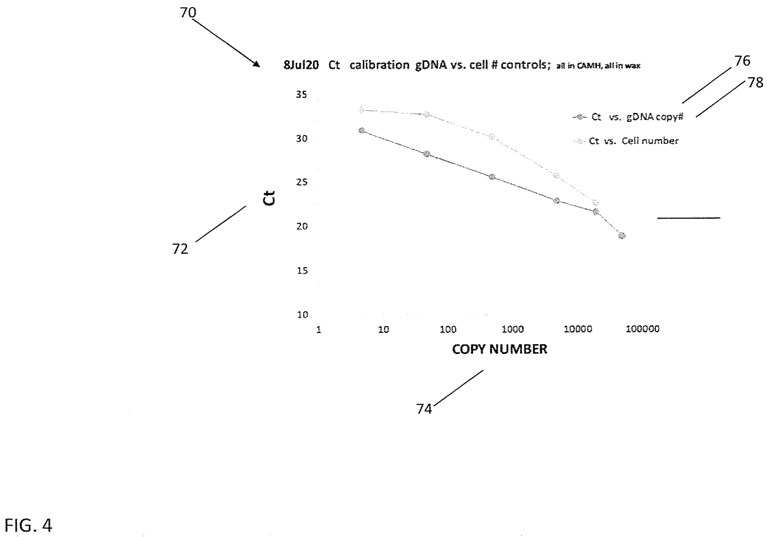

With the foregoing in mind, FIG. 4 is a graph 70 of bacteria growth using the lyophilized reagent drug bead, in accordance with embodiments of the present disclosure. The graph 70 represents a copy number of DNA 74 on the x-axis, and a Ct count 72 on the y-axis resulting from the cell lysis phase 40 of AST testing. During the cell lysis phase 40 amplification reagents are released as a result of increasing the temperature of the amplification reaction vessel 24 that melts the solid wax 28 that includes the lyophilized amplification bead 20. The bacteria growth testing was graphed according to Ct count versus gDNA copy number 74, and graphed according to Ct count versus the cell number 78 over time.

For lyophilized reagent beads preparation, Hemo KlenTaq was purchased from New England Biolabs as a glycerol free solution. Tetracycline hydrochloride was purchased from Sigma. Forward and reverse amplification primers were purchased from Integrated DNA Technologies, Inc. EvaGreen and ROX normalizing dye were purchased from Biotium. All other chemicals were purchased from Sigma-Aldrich.

Lyophilized reagent beads were prepared such that dissolution of each bead in 10 μL of water gave a solution with 60 mM Tricene, pH=8.75, 5 mM ammonium sulfate, 3.5 mM magnesium chloride, 0.5 mM dNTPs (0.5 mM each: dATP, dCTP, dGTP, dTTP), 0.75 μM forward (5′-GAA GAG TTT GAT CAT GGC TCA GAT TG-3′) and 0.75 μM reverse (5′-TTA CTC ACC CGT CCG CCA C-3′) primers, 150 U Hemo KlenTaq in 10 mM KCl, 1 mM Tris-HCl, pH=7.4, 0.01 mM EDTA, 0.1 mM dithiothreitol, 0.05% Tween 20, and 0.05 IGEPAL CA-630 (octylphenoxypolyethoxyethanol), 0.0625 μM Eva Green in 0.5 mM Tris, pH=8.0, 5 μM EDTA, and 5×10-5% Tween 20, 1 μM ROX 25 μM ROX normalizing dye.

Tetracycline lyophilized reagent beads were prepared such that dissolution of each bead in 10 μL of water yielded a solution containing 0.1, 0.5, 2, and 10 μg tetracycline/mL. For antimicrobial susceptibility testing in the single phase assay, the potency of lyophilized beads of tetracycline hydrochloride against E. coli was tested using cell growth indicator- and amplification-based assays and compared with the results obtained from same assays performed in the presence of liquid form of the antibiotic. Minimum inhibitory concentrations (MIC) value for tetracycline against E. coli was determined by microtiter plate-based antibacterial assay incorporating alamarBlue as an indicator of cell growth. Briefly, each well in a 96-well plate contained 100 μL mixture of 10 μL of 500 E. coli cells/μL (ATCC 25922) suspended in cation-adjusted Mueller Hinton broth (CAMHB), 50 μL of 1:1 serial dilution of tetracycline in CAMHB, 10 μL of alamarBlue HS Cell Viability Reagent (Thermo Fisher, A50100) and 30 μL growth medium of CAMHB. For a positive growth control, the antibiotic was replaced with CAMHB, and sterility (no growth) control included solutions without any cells. The cells were then grown in the presence of different concentrations of liquid tetracycline including controls for 16 hours at 37° C. without any agitation. Alternately, the cells were grown in the presence of lyophilized beads consisting of different concentrations of tetracycline or a drug-free bead (positive control) under similar conditions as the liquid drug in tubes. The 96-well plate was then imaged for any color change in the wells to assess the bactericidal activity of tetracycline at different concentrations and determine the corresponding MIC value.

Titration of tetracycline in amplification Assay: 10 μL of E. coli culture containing 5000 cells were grown for 3 hours at 37° C. in the presence of a serial dilution of liquid tetracycline or no drug as described above. After this growth phase, 1 μL aliquot of the culture was added to an amplification tube containing 10 μL of amplification mixture. The details of the amplification mixture and primer information were as described earlier. The Ct values obtained were analyzed to estimate the MIC value and compare them with the MIC values obtained from Alamar Blue assay.

A multi-phase assay for AST involved two 10 μL LRB in a single 0.1 mL amplification tube (FIG. 1)—one bead consisting of tetracycline at the desired concentration and the other consisting of the amplification mixture. The paraffin wax volume was optimized to be ˜10 μL and was measured using mass/density information from the vendor (Sigma-Aldrich Cat. #411663-1KG). The weight of the wax was punched from a molten sheet of solid wax and added to an amplification tube and melted by raising the temperature to just above 65° C. Next, the amplification bead is immediately embedded in the wax at the bottom of the amplification tube by dropping it in the molten wax and letting it cool down to room temperature. After the wax solidification, the drug bead (or no-drug control bead) was placed on top of the solid wax. 10 μL of E. coli culture containing 5000 cells were introduced inside the tube and incubated at 37° C. for 3 hours in the absence of any agitation as described above. The same tube is then placed in the amplification instrument to initiate the qPCR. The corresponding Ct values were analyzed to estimate the MIC value and compare them with the MIC values obtained from single phase assays (amplification and alamarBlue).

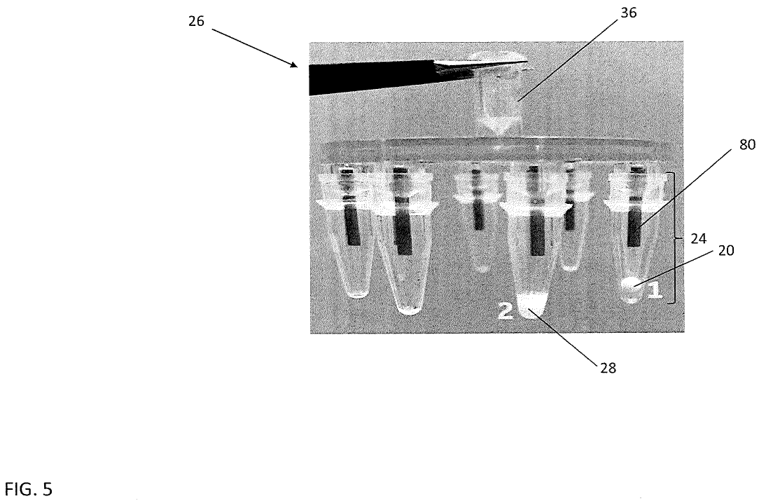

As discussed above, during transfer of sample to the amplification reaction vessels 24 the microfluidic manifold 26 device may be used to enable transfer of sample 30 to the amplification reaction vessels 24 in a closed system environment. FIG. 5 is an image of the microfluidic manifold 26 that includes one or more samples 30 with the amplification bead 20, in accordance with embodiments of the present disclosure. The microfluidic manifold 26 may enable equal distribution of the input sample 30 (e.g., bacterial sample) into eight amplification reaction vessels 24, or any suitable number of amplification reaction vessels 24 attached to each of the eight outputs shown within the microfluidic manifold 26.

The microfluidic manifold 26 may be fabricated using a clear material (e.g., acrylonitrile butadiene styrene (ABS)), and may be manufactured using 3-D printing techniques at a resolution of approximately 100 micro-meters, or any other suitable manufacturing technique. The eight amplification reaction vessels 24 may attach to each connecting channel 80 component of the microfluidic manifold 26, and the sample 30 may be input at the sample inlet 36 of the microfluidic manifold 26. The sample 30 may be input at different concentrations into each of the amplification reaction vessels 24. The amplification reaction vessels 24 may each contain the amplification bead 20, the amplification bead 20 embedded within the solid wax 28, or any other amplification reagent format.

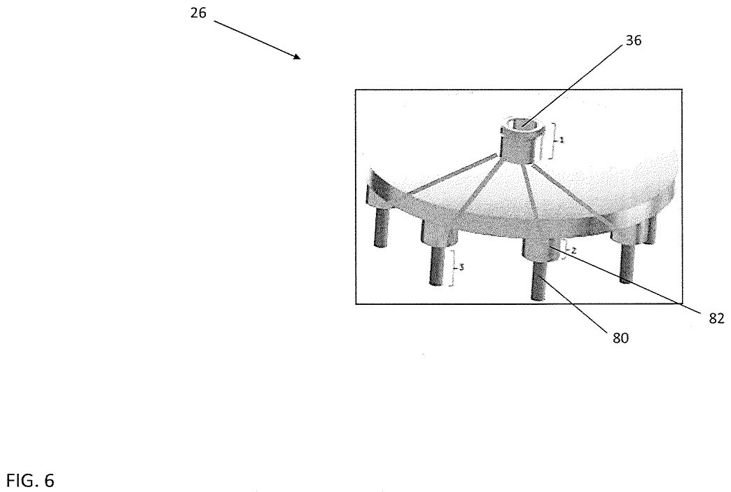

Keeping the foregoing in mind, FIG. 6 is a schematic diagram of the microfluidic manifold 26, in accordance with embodiments of the present disclosure. As discussed, above the microfluidic manifold 26 may be used to ensure even sample distribution into amplification vials during MIC and AST testing and analysis.

The microfluidic manifold 26 may include a sample inlet 36 compatible with standard syringes (or liquid dispensing devices) with a locking interface. The microfluidic manifold 26 may include eight or any suitable number of radially split connecting channels 80 used for distribution of the sample from the sample inlet 36, to the outlets that connect to the amplification reaction vessels 24. The terminal end of each channel includes a round connector 82 that enables a tight connection between the amplification reaction vessels 24 and the connecting channels 80. In some embodiments the amplification vials may include a venting opening component. The connecting channels 80 within the microfluidic manifold 26 may be made from polyetheretherketone (PEEK), or any other suitable material with an inner diameter of a small length (e.g., 0.125 micro meters) and a greater length than inner diameter (e.g., 1 cm). The connecting channels 80 may be attached to the round connector 82 at the end of the internal connecting channels 80. The PEEK tubing's may be secured by fitting into one or more grooves of the round connectors 82, and may permanently attached using plastic epoxy. The microfluidic manifold 26 may be sterilized prior to each use by exposing each side of the microfluidic manifold 26 to ultraviolent light for at least thirty seconds on each side, or any suitable time amount.

During sample distribution with the microfluidic manifold 26 the amplification reaction vessels 24 may be attached to each of the individual round connectors 82 of the connecting channels 80. The sample 30 may then be pipetted or transferred through other suitable transfer device into the sample inlet 36 of the microfluidic manifold 26. A syringe may then be placed into the sample inlet 36 and actuated to displace the sample 30 from the sample inlet 36 into each of the connecting channels 80 that are connected to each of the amplification reaction vessels 24. The amplification reaction vessels 24 may then be detached and processed for AST and amplification assay.

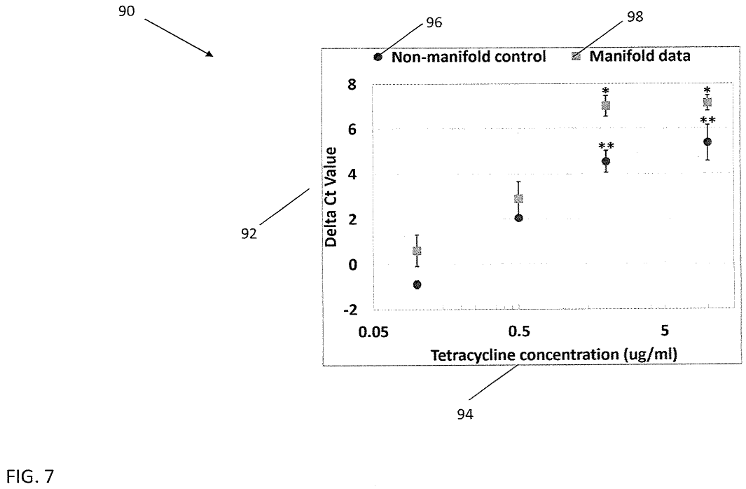

Keeping the foregoing in mind FIG. 7 is a graph 90 of tetracycline concentration versus delta Ct values to demonstrate microfluidic manifold sample retention, in accordance with embodiments of the present disclosure. Amplification was performed using the microfluidic manifold 98 during the sample 30 distribution, and also using non-manifold control methods 96 for the sample 30 distribution during amplification. The non-manifold control 96 methods include pipetting samples directly into each amplification reaction vessel 24 that is to be tested.

The results of the amplification analysis for the non-manifold control method 96 of sample distribution and the microfluidic manifold 26 sample distribution method were graphed. The y-axis corresponds to the delta Ct values 02 resulting from both sample distribution methods and the x-axis corresponds to the tetracycline concentration 94 of the samples 30. The delta Ct values 92 were obtained by using the Ct values collected using the control bead and subtracting them from the Ct values 92 obtained in the presence of an antibiotic bead 22. Both the microfluidic manifold 26 loaded samples 30 and non-manifold control 96 the manually loaded samples 30 resulted in demonstrated growth inhibition at the same concentrations of the tetracycline antibiotic, as shown by the graph 90. Statistical ANOVA tests were conducted for both data sets, and the range of deviation for each data point was graphed. It should be understood, that the microfluidic manifold 98 produced results that demonstrate improved sample 30 retention performance over traditional non-manifold methods.

Technical effects of the disclosure include phenotypic antimicrobial susceptibility testing information by pairing short growth periods (depending on the organism and typically in the range of a few hours, for example 3-4 hours) with downstream amplification assays to predict minimum inhibitory concentration values of antibiotics for treatment. The disclosed techniques streamline the potentially complex nature of workflows (i.e., number of steps required, consumables utilized, manual intervention per step, sample loss/damage during transfer and handling etc.) required to execute these assays into a single-vessel format capable of hosting both reactions. While antimicrobial susceptibility testing requires the growth of bacteria at 37° C. in the presence of physiologically relevant buffers and pH, nucleic acid amplification assays (typically amplification) are performed at least in part at higher temperatures with very specific pH and reagent compositions (salt, media etc.) to support uninhibited nucleic acid amplification and detection. The disclosed techniques address the mismatch in reaction conditions by providing a reaction vessel with spatial segregation of these chemistries for effective implementation of both assays in a single-vessel format for reliable minimum inhibitory concentration analysis. In embodiments, the disclosed techniques may be used in conjunction with devices configured for automated reporting or display of amplification results to generate a notification of antibiotic susceptibility of the biological samples that are tested. Accordingly, the disclosed techniques may include systems for antibiotic susceptibility testing. In addition, the disclosed techniques may include kits or components, e.g., disposable or consumable components, for carrying out antibiotic susceptibility testing. In certain embodiments, individual components may be provided. In an embodiment, an amplification bead or an antibiotic bead may be provided as separate components with or without other kit elements.

This written description uses examples, including the best mode, and also to enable any person skilled in the art to practice the disclosed embodiment, including making and using any devices or systems and performing any incorporated methods. The patentable scope is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A method of antibiotic susceptibility testing comprising:
providing a lyophilized reagent bead comprising amplification reagents, wherein the lyophilized reagent bead is embedded within a first layer of a reaction vessel, wherein the first layer comprises a phase change material having a melting point temperature;
providing an antibiotic bead comprising one or more antibiotics to the reaction vessel while the first layer is solid;
adding a bacterial sample to the reaction vessel to contact the antibiotic bead in a second layer;
incubating contents of the reaction vessel at a temperature below the melting point temperature and to release the one or more antibiotics of the antibiotic bead in the second layer;
increasing a temperature of the contents of the reaction vessel to be above the melting point temperature to melt the phase change material to permit the bacterial sample to contact the lyophilized reagent bead; and
conducting amplification analysis of the bacterial sample using amplification reagents of the lyophilized reagent bead.

2. The method of claim 1, wherein the sample is distributed to one or more reaction vessels through insertion into a sample inlet of a microfluidic manifold device, wherein the microfluidic manifold device comprises one or more internal channels connecting the sample inlet to a plurality of outlets of the microfluidic manifold device.

3. The method of claim 2, wherein adding the bacterial sample to the reaction vessel comprises inserting a syringe or liquid dispensing device into the sample inlet of the microfluidic manifold device, wherein the syringe comprises the bacterial sample, and actuating the syringe to insert the sample.

4. The method of claim 2, wherein each of the plurality of outlets of the microfluidic manifold device comprise an interlocking mechanism that receives a respective reaction vessel to couple to the microfluidic manifold.

5. The method of claim 3, wherein each interlocking mechanism comprises a venting hole.

6. The method of claim 1, wherein a growth medium is added to the reaction vessel prior to increasing the temperature.

7. The method of claim 1, wherein a dried growth medium is provided in the reaction vessel, wherein the dried growth medium dissolves upon contact with the bacterial sample.

8. The method of claim 1, wherein the PCR lyophilized reagent bead and the antibiotic bead each comprise a spherical-shaped particle.

9. The method of claim 1, wherein adding the bacterial sample to contact the antibiotic bead causes the antibiotic bead to dissolve.

10. The method of claim 1, wherein the second layer is liquid while the first layer is solid at temperatures below about 65° C.

11. A method of antibiotic susceptibility testing comprising:
providing a reaction vessel comprising:
an amplification bead comprising lyophilized amplification reagents;
a solid layer; and
an antibiotic bead comprising one or more lyophilized antibiotics,
wherein the antibiotic bead is separated from the amplification bead by the solid layer;
adding a bacterial sample to the reaction vessel to contact the antibiotic bead in a second layer to at least partially dissolve the antibiotic bead and form a liquid layer comprising the bacterial sample and the at least partially dissolved antibiotic bead separate from the solid layer;
melting the solid layer to cause displacement of the liquid layer and such that the amplification bead is in the liquid layer; and
conducting an amplification assay of the bacterial sample using the amplification reagents of the amplification bead.

12. The method of claim 11, wherein the reaction vessel comprises a bacterial growth medium.

13. The method of claim 11, comprising incubating the reaction vessel at a bacterial growth temperature prior to melting the solid layer.

14. The method of claim 11, wherein the amplification bead is embedded in the solid layer, and wherein the solid layer is at a bottom of the reaction vessel and the liquid layer is atop and in direct contact with the solid layer prior to melting the solid layer.

15. The method of claim 14, wherein melting the solid layer causes the melted layer to displace the liquid layer into the bottom of the reaction vessel.

16. The method of claim 15, wherein the melted layer is above and in direct contact with the liquid layer.

17. The method of claim 16, wherein the amplification bead is separated from the melted layer.

18. An antibiotic susceptibility testing reaction vessel comprising:
an amplification bead comprising lyophilized amplification reagents embedded within a solid layer, wherein the solid layer is solid below 37° C. and has a melting point above about 65° C.; and
an antibiotic bead comprising one or more lyophilized antibiotics.

19. The reaction vessel of claim 18, wherein the reaction vessel comprises an end configured to fluidically couple to a sample inlet of a microfluidic manifold.

20. The reaction vessel of claim 18, wherein the solid layer is on a bottom of the reaction vessel and wherein the solid layer separates the antibiotic bead and the amplification bead from direct contact from one another at temperatures below the melting point.

21. The reaction vessel of claim 18, comprising a biological sample in contact with the antibiotic bead.

22. A method of antimicrobial testing:
providing a lyophilized antibiotic bead comprising one or more lyophilized antibiotics having a lyophilized shell that is stable at 37° C. and configured to dissolve when contacted by a liquid bacterial sample, wherein components of the lyophilized antibiotic bead do not inhibit an amplification assay of the liquid bacterial sample when mixed with additional reagents of the amplification assay.
